(19) United States
(12) Patent Application Publication
(10) Pub. No.: US 2016/0303037 A1
(43) Pub. Date: Oct. 20, 2016

(54) COMPOSITION FOR A PERFUME DELIVERY SYSTEM HAVING A TRIPLE-ACTION AND A BI-MODAL RELEASE

(71) Applicants: HEMANT NARAHAR JOSHI, Parsippany, NJ (US); Harshada N. Sant, Harrison, NJ (US)

(72) Inventors: HEMANT NARAHAR JOSHI, Parsippany, NJ (US); Harshada N. Sant, Harrison, NJ (US)

(21) Appl. No.: 14/687,046

(22) Filed: Apr. 15, 2015

(51) Int. Cl. A61K 8/98; A61K 8/34; A61Q 19/00; A61K 8/73; A61K 8/97; A61Q 13/00; A61K 8/39; A61K 8/37

(52) U.S. Cl. CPC A61K 8/981; A61K 8/39; A61K 8/345; A61K 8/37; A61K 8/738; A61K 8/97; A61Q 13/00; A61Q 19/007

(57) ABSTRACT

Compositions are herewith provided for a triple action perfume delivery system with a biphasic release of fragrances. The carrier system provides a moisturizing and emollient effect to the skin. It releases the fragrances over a long period of time. More importantly, a portion of perfume is released after sweating (sweat-activated action).

### FIELD OF INVENTION

The present invention relates to compositions of perfumes, which are moisturizing/emollient, long-lasting and sweat-activated. The fragrances show a bimodal release.

### BACKGROUND OF INVENTION

In simplest terms, in current era, a perfume is a mixture various scents. The art of making perfumes was developed by Egyptians and later adopted by Romans, Persians, and Indians etc. Early Egyptians used perfumed balms as part of religious ceremonies and later as a part of pre-love making preparations. The key ingredient of a perfume is a specially denatured alcohol, which constitute to about 78% to 95%. The remainder portion is a mixture of essential oils, fragrances or scents. Perfume is the costliest form of fragrance with >22% of essential oils. Eau de Parfum (EDP) comes next with between 15 and 22% essential oils followed by Eau de Toilette (EDT) with 8 to 15% oils. The weaker Eau de Cologne has just ˜4% essential oils. For those who crave super subtlety, Eau Fraiche with 1 to 3% essential oils is the lightest dilution of fragrance.

Fine fragrances can be categorized to several different aroma groupings. All scents, no matter how unique and distinct, fall into fragrance families that include floral, chypre, oriental, lavender, fougere and citrus. They are further divided between men and women.

Women:

1. Chypre—It is based on a woody, mossy, floral accord, which can include leathery or fruity notes as well. Chypre perfumes have a rich and lingering scent. The compositions are based on oakmoss, ciste-labdanun, patchouli and bergamot accords. The richness of chypre notes mixes wonderfully with fruity or floral notes.

2. Citrus—Each perfume in this family is primarily composed of citrus scents such as bergamot, lemon, orange, tangerine and grapefruit, to which other orange-tree elements (orange blossoms, petit grain or neroli oil) have been added. These perfumes are characterized by their freshness and lightness. The citrus accord is enhanced by the addition of aromatic notes, such as thyme, rosemary, tarragon or mint.

3. Floral—This family is composed of a large variety of creations ranging from sumptuous bouquet arrangements to “soli flora’ compositions. The floral note can be enriched with green, aldehydic, fruity or spicy hints. With its natural scent, the floral note is one of the most widely used in women's perfumes.

4. Oriental—These notes also known as “amber” notes—stand out because of their unique blend of warmth and sensuality. They draw their richness from heady substances like musk, vanilla and precious woods, often associated with exotic floral and spicy scents.

Men

1. Aromatic—Aromatic notes are mainly composed of sage, rosemary, thyme and lavender usually complemented with citrus and spicy notes. These notes are often used in men's fragrances. 2. Citrus—This family includes all fragrances mainly composed of citrus notes such as bergamot, lemon, orange, tangerine and grapefruit. These fragrances are characterized by their freshness and lightness. The masculine character comes from the frequently strong presence of aromatic and spicy notes.

3. Oriental—Refreshed by aromatic or citrus facets, oriental compositions draw their richness and sophistication from precious substances such as amber, resin, tobacco, spices, exotic woods and animal notes.

4. Woody—These fragrances with their woody middle note are warm and opulent when based on sandalwood or patchouli. Cedar and vetiver make them dryer. These warm, dry and elegant masculine accords often contain a dash of citrus or aromatic notes.

Perfumes can also be classified as different notes—Top, Middle and Base. The underlining property is their evaporation rates.

The Top Notes—These are sometimes referred to as the opening notes or head notes. The top notes of a fragrance are generally the lightest of all the notes. They are recognized immediately upon application of the perfume. The top notes are also the first to fade due to their higher evaporation rates. But this does not mean they aren't of utmost importance. It is hugely important that the top notes smoothly transition into the heart of the fragrance. Common fragrance top notes include citrus (lemon, orange zest, bergamot), light fruits (grapefruit, berries) and herbs (clary sage, lavender).

The Middle Notes—The middle notes, or the heart notes, make an appearance once the top notes evaporate. They last longer than the top notes and have a strong influence on the base notes to come. A perfume's heart is generally pleasant and well-rounded. It is often a smooth combination of floral or fruit tones; sometimes infused with spices like cinnamon, nutmeg or cardamom. Common fragrance middle notes include geranium, rose, lemongrass, ylang ylang, lavender, coriander, nutmeg, neroli and jasmine.

The Base Notes—The base notes are the final fragrance notes that appear once the top and middle notes are completely evaporated. The base notes mingle with the heart notes to create the full body of the fragrance, but are typically associated with the dry-down period. In this patent, the dry-down period is defined as the actual time it takes to completely dry down the applied perfume formulation on a surface. The job of the base notes is to provide the lasting impression. These often rich notes linger on the skin for hours after the top and middle notes have dissipated. Common fragrance base notes include cedarwood, sandalwood, vanilla, amber, patchouli, oak moss and musk. Without the combination of the three levels of notes, a fragrance just wouldn't be aromatically appealing. But together, they create beautiful scents.

Wearing a perfume is more of an art than science and it can be very expensive. For example, in layering of perfumes, one uses a similar perfume in the body soap/shampoo during shower followed by one in the lotion, then in the body spray and lastly as an Eau de Parfum. One may apply perfumes to pulse points or spray in air and then walk slowly in it.

Till 1960s, only rich people could afford good perfumes. In recent decades, the perfume industry has evolved and now common people have started buying perfumes regularly. In early 60s, most of innovations were towards coming up newer kinds of perfumes and many new fragrances were invented. In 90s, attention was diverted to preparing different kinds of perfume carriers. In the U.S. Pat. No. 8,343,521, inventors eliminated ethyl alcohol from their perfume compositions and used 1,2-hexanediol or 1,2-heptanediol with water as a solvent system. U.S. Pat. No. 6,620,437 described a perfume containing water-in-oil microemulsion. U.S. Pat. No. 8,889,614 used encapsulation technique for perfume compositions. An aqueous aerosol composition was used in the U.S. Pat. No. 8,865,132.

In the U.S. Pat. No. 8,895,041, inventors developed a skin body wash or cleansing formulation which contained at least 5%, by weight of the composition, of a surfactant, b. at least about 25%, by weight of the composition, of water, and c. a cyclodextrin complex comprising a perfume. The purpose was to deliver perfumes more effectively and should be long-lasting beyond the initial cleansing of the user. The fractional delivery value of at least 0.69 was obtained. The degree of complexation of at least 90% was used prior to incorporation into the cleansing composition. The ratio of water to cyclodextrin complex is between about 15:1 and 1:1 within the body wash composition. In the current patent application, water was not added as a component in any of the formulations developed.

Recently, in a research article, authors described a water-triggered release of fragrances (Chem. Commun. 51:4455-4457, 2015). Authors prepared pro-fragrances, similar to prodrugs. The fragrance was released by hydrolysis of pro-fragrance with water. U.S. Pat. No. 9,000,052, described a perfuming composition capable of prolonging the release of perfuming components with the use of hydrophobic block copolymers. The long-lasting effect was achieved by slowing the diffusion of perfuming compounds through copolymers. In another recent U.S. Pat. No. 8,992,889, a freshening composition having a malodor control component in a hydro alcoholic mixture was provided. The malodor components were a mixture of volatile aldehydes. Inventors claimed the use of uncomplexed cyclodextrins—the exact function had not been described. The % cyclodextrin in the formulations ranged from 0.15 to 1.5% and might have been used as a solubilizer.

Perfume is a generic term which includes fragrances or scents. For the purpose of this patent application, a mixture of various fragrances forms a perfume formulation. Various types of products include perfumes such as body washes, laundry detergents, foams, creams/lotions, balms, scented oils, deodorant and commonly used spray perfumes. This patent application is mainly for the perfumes to be applied to body, hair, and clothes or to be spread in air.

The underarm areas (axillae) are the parts of the body that perspire the most and so contribute most to the body odor. Whether pleasant or unpleasant, body odor is the result of a mixture of volatile chemical substances from underarm perspiration—steroid hormones, fatty acids and sulfur-containing compounds. These react when they come into contact with the microbial flora of the skin.

The flora contains bacteria from a wide variety of species, such as staphylococcus or coryneform bacteria, whose main function is to protect and nourish the layers of the skin. In coexisting in varying amounts and combinations in our skin, these microbes leave a different trace in each person. They transform non-odorous natural secretions into volatile molecules that smell. Various combinations between the characteristics of our skin, the environment and perfume molecules create an endless number of smells, each virtually unique.

When external fragrances are mixed with the cocktail of skin molecules, they trigger a series of chemical reactions that produce a unique smell. The quality and intensity of the smell depends on the amount of fragrance the skin absorbs and how much evaporates. Wearing clothes over perfumed skin, product absorption and less evaporation can be ensured.

The degree of moisture emitted by the pores of the skin can also influence the amount of fragrance that evaporates. Perfume applied to well-hydrated skin that produces a lot of moisture in a dry room at a high temperature evaporate more quickly.

Perfume molecules, which dissolve when mixed with fatty acids, also interact with other epidermal fats (ceramides and cholesterol). These substances, present in individual skin in varying amounts and proportions, are the first barrier to perfume being absorbed through the skin.

Based on this discussion, it is clear that perfumes are complex by themselves. But the outcome from perfumes once applied could be different for different people. The performance of perfume formulation will also vary depending on the external environmental conditions such as heat and moisture in air.

The objective of this invention is to provide compositions which would act as a perfume delivery system that would have three distinct effects —moisturize and soften the skin, release the perfume over a long period and some portion of perfume should be released only upon sweating, i.e., sweat-activation. The top notes are allowed to evaporate slowly compared to when dissolved only in the Perfumers alcohol, making them long-lasting. This perfume delivery system will also show a bimodal release profile. There could be formulation compositions having one or two of the three effects mentioned in this paragraph.

### SUMMARY OF INVENTION

The main objective of this product was to develop a perfume delivery system, which can be applicable to any perfume product irrespective of the combination of the fragrances in the perfume. This perfume delivery system has been designed to have key three actions—moisturizing/emollient, long-lasting, and sweat-activated. Thus, one of the objectives of the invention is to provide a perfume composition that is sweat-activated. This can be accomplished by the use of cyclodextrins. Cyclodextrins are bucket-shaped molecules, which can entrap the fragrance molecules in their bucket shaped structure. In dry state, the fragrance molecules are complexed with cyclodextrin and are not released. When in contact with moisture or sweat, the cyclodextrin molecules dissolve making them mobile. It helps to release the fragrance molecules entrapped in the cyclodextrin cavity. Sweating provides moisture needed for the release of fragrance from the cyclodextrin cavity.

In another embodiment, Moisturizing agents/humectants are used to give a smooth feel to the skin. Perfumer's alcohol (major ingredient is 200 proof ethanol or ethyl alcohol) can cause dryness of the skin when spread. To counteract this dryness, humectants or moisturizing agents are added so that they can lock in the moisture into the skin.

Another objective of the invention is make it long lasting. This effect is achieved by emollients and to some extent, with cyclodextrins. Fragrances dissolve in the emollients added to the delivery system. Emollients get entrapped in the epidermal layers and thereby slowly release the fragrance.

Another objective of the invention is to convert part of the top notes to middle or base notes by slowing down their evaporation rates upon application. Most of the emollients would slow down the release of fragrances. Moisturizing agents also have a similar effect to a lesser extent.

Another objective of the invention is to upload the liquid perfume with desired properties on a solid powder base such as calcium silicate and talc, or mixtures thereof, to produce a scented powder.

Another solution further consisted in providing perfume compositions in which the ethanol is at least partially replaced by one or more volatile silicones which are linear-of the polydimethylsiloxane type. Another objective of the invention is to replace part of perfumer's alcohol used in the formulations by 1,2-hexanediol or 1,2-heptanediol or mixtures thereof.

One of the objective of the perfume composition is to apply to objects such as a living body surfaces, clothing, hair, etc. Another objective of the perfume composition is to apply to non-living objects such as curtains, artificial floral arrangement, Christmas trees etc.

### BRIEF DESCRIPTION OF THE DRAWINGS

FIG. 1 shows the results of evaporation of perfumer's alcohol at room temperature. FIG. 1 also shows the results of evaporation of a model perfume, manzanate, in the presence of alcohol and a cyclodextrin at room temperature.

FIG. 2 shows the results of evaporation of a model perfume (manzanate) in the presence of alcohol, in alcohol with propylene glycol and in alcohol with isopropyl myristate.

### DETAILED DESCRIPTION

### Definition of Terms Used

Unless defined otherwise, all technical and scientific terms used herein have the same meanings as commonly understood by one of ordinary skill in the art.

As used herein and in the claims, the singular forms “a”, “an”, and “the” include plural reference unless the context clearly dictates otherwise. For example, reference to “an excipient” is a reference to one or more excipients and equivalents thereof known to those skilled in the art.

The term “about” is used to indicate that a value includes the standard level of error for the substance or method being employed to determine the value. The terms “comprise,” “have” and “include” are open-ended linking verbs. Any forms or tenses of one or more of these verbs, such as “comprises,” “comprising,” “has,” “having,” “includes” and “including,” are also open-ended.

“Excipients” are compounds used in the formulation along with the active ingredients, i.e., perfumes in this application. The active has to stable in the formulation along with the excipients throughout the shelf-life.

“Pharmaceutically Acceptable Materials” refers to those compounds or materials, which are suitable for use in contact with tissues or organs of humans and animals without excessive toxicity, irritation, allergic response or any other problems. Only pharmaceutically acceptable excipients were used in the current formulations in this patent application as the perfumes are targeted to be applied to human skin.

“Chemical stability” with respect to the active agent means that an acceptable percentage of degradation products are produced by chemical pathways such as hydrolysis, thermal degradation or oxidation during the shelf-life of the product. In this application, the active agents are the fragrances used in the perfume formulations. An active ingredient is a compound, which imparts the aroma to the perfume compositions.

“Physical stability” with respect to the active agent means that an acceptable percentage of aggregates, loss of original color or discoloration, crystals, visible mold/fungus is formed during storage of the perfume.

The perfume formulation is considered “stable” when it shows good chemical and physical stability.

The word ‘perfume’ itself derives from the Latin word per which means through and fumare which means smoke. It means, evaporation is a critical property of a perfume.

Eau de Cologne—It is delightfully refreshing in hot weather and it doesn't last long. It needs to be applied frequently and tends to be marketed in large sizes of up to 200 ml and is often applied by spray. Eau de Cologne or simply cologne (German: “Water of Cologne”) is a perfume originating from Cologne, Germany. Eau de cologne contains a mixture of citrus oils including oils of lemon, orange, tangerine, bergamot, lime, grapefruit and neroli. It can also contain oils of lavender, rosemary, thyme, petitgrain (orange leaf), jasmine, and tobacco. It has 70-90% ethanol and remaining water.

Eau de toilette—Sometimes used to describe the same concentration as cologne. The top notes are dominant, making it refreshing when it is applied, and it evaporates and fades away quite quickly. Eau de toilette and cologne are the most popular forms, and are particularly appropriate for the summer.

Eau de parfum—After the top notes have died away, the middle notes or heart notes of a perfume become noticeable. This is the focus of Eau de parfum, which makes it perfect to spray on hair or clothing.

Perfume—Also called perfume extract or extrait, this is the most expensive version of any fragrance; it's also the most beautiful because of the whole symphony of top, heart and base notes released over a period of time. Perfume is applied directly to the skin on pulse spots—insides of the wrists, behind the ears, at the throat.

As the name suggests, a “delivery system” is a system or composition to deliver a product to living or non-living objects. In pharmaceuticals, it is a device for administering drugs to patients. In cosmetics, a perfume delivery system is a system for administration the perfume to the user and people around it. Cosmetics are generally applied topically. There are two aspects —actual application by the user on the skin or on clothes. Once applied, the fragrance should be carried from the skin or clothes to air surrounding the user and people around should smell the perfume. The extent and the rate of evaporation of perfume are critical. Carrier in general means a system which carries an object from point A to point B. In perfumes, carrier carries the perfume from the primary container to the surface (skin or clothes or air) and from the surface to the user or people coming in contact with the user.

“Fixatives” are used to equalize the vapor pressures, and thus the volatilities, of the raw materials in perfume oil, as well as to increase the tenacity. Dipropylene glycol and diethyl phthalate are good fixatives. Other commonly used fixatives are—Hedione, Abalyn™ D-E Methyl Ester of Rosin, Floraesters K-100 Jojoba, Floraesters K-20W Jojoba, Sepicide LDForalyn 5020-F, CG Hydrogenated Rosinate.

“Emollients” are bland, fatty, or oleaginous substances, which increase tissue moisture content thereby rendering skin softer and pliable. The increase in water content can be achieved by preventing water-loss or increasing water holding capacity. Application of emollients prevent water-loss from skin whereas humectants increase the water holding capacity of the skin. The emollients can be animal fats/oils, vegetable oils, and waxes. Commonly used emollients are: Natural—cold-pressed almond oil, jojoba oil, sunflower oil, olive oil, hazelnut oil, avocado oil, safflower oil, wheat germ oil, apricot kernel oil, and natural waxes such as unrefined beeswax, shea butter and cocoa butter, Synthetic—Schercemol™ LL Ester, Schercemol™ 1818 Ester, butylene glycol, capric/caprylic triglyceride, ceteareth-20, fatty alcohols (e.g., cetearyl alcohol, cetyl alcohol, coconut fatty acids), silicones (e.g., cyclomethicone, dimethicone, cyclopentasiloxane), emulsifying wax, petroleum jelly, fatty acids, glyceryl stearate, hydrogenated oils, isopropyl myristate, jojoba butter (hydrogenated jojoba oil), mineral oil, octyl palmitate, paraffin, squalene, stearic acid, palmitoyl proline, magnesium palmitoyl glutamate, ethyl hexyl hexanoate, diisopropyl adipate, glyceryl triisononanoate, nonoxynol-2, polydecene, olive oil PEG-7 esters, cetearyl octanoate, 18-Dimethicone, PEG-12 Dimethicone, butylene glycol, caprylic triglyceride, cetareth 20, cyclomethicone, cyclopentasiloxane, Sodium palmitoyl sarcosinate, coco caprylate, xylitylglucoside, anhydroxyxylitol, ethyl oleate, triethylhexanoin, isocetyl stearate, isostearyl isostearate, myristyl myristate, pentaerythrityl tetracarylate, cetyl palmitate etc.

“Humectants” are substances introduced into the stratum corneum to increase its water holding capacity. Commonly used humectants are —propylene glycol, hexylene glycol, and butylene glycol, glyceryl triacetate, neoagarobiose, sugar alcohols (sugar polyols) such as glycerol, sorbitol, xylitol, maltitol, polymeric polyols such as polydextrose, quillaia, urea, aloe vera gel, alpha hydroxy acids such as lactic acid, honey.

“Bridging agent”—as the name suggests, it forms a bridge between the formulation components and the skin layer or the surface such as fibers on which the perfume has been applied.

As used herein, the term “cyclodextrin” includes any of the known cyclodextrins such as unsubstituted cyclodextrins containing from six to twelve glucose units, especially, alpha-, beta-, and gamma-cyclodextrins, and/or their derivatives, and/or mixtures thereof. The alpha-cyclodextrin consists of 6, the beta-cyclodextrin 7, and the gamma-cyclodextrin 8 glucose units arranged in a donut-shaped ring. The specific coupling and conformation of the glucose units give the cyclodextrins a rigid, conical molecular structure with a hollow interior of a specific volume. The “lining” of the internal cavity is formed by hydrogen atoms and glycosidic bridging oxygen atoms; therefore this surface is fairly hydrophobic. These cavities can be filled with all or a portion of an organic molecule with suitable size to form an “inclusion complex.” Such entrapment can affect the volatility, solubility or chemical stability of complexing molecules.

“Sweat-activated” is a system, which is activated in the presence of sweat. In this patent application, the perfume delivery system is sweat-activated. The sweat is predominantly made up of water. Thus, sweat-activated system can also be a water-activated system. This sweat-activated system releases fragrances when it comes in contact with the sweat. It may release the fragrance after coming in contact with any other source of water. The moisture in air can trigger the release of perfume as well. In short, there should be enough water for the cyclodextrin to get dissolved and release the entrapped fragrance molecule from its cavity.

A “Powder Base” for the purpose of this patent application is a mixture of powders to be applied topically. A typical powder base contains Talcum or corn starch. The powder base may also contain other ingredients such as calcium silicate, silica or glitters or pigments.

A “Solvent” for the purpose of this patent is the diluent used. Ethyl alcohol is most commonly used at the “solvent” for the perfume formulations. Perfumers Alcohol is prepared from 200 proof ethyl alcohol which may contain very small amounts of butyl alcohol, denatonium benzoate (Britex), and hexylene glycol. Various grades of Perfumers Alcohol are available. Commonly used grades are SDA 40B 200 Proof and SDA-B 200 proof. Other solvents such as, 1,2-hexanediol or 1,2-heptanediol or volatile silicones, can be used in place of or with Perfumers Alcohol.

These perfume compositions may be intended for application to a living body, clothing, hair, etc. For example, a flower fragrance may be applied to a person. These perfume compositions may also be used on non-living objects such as window curtains, artificial flower arrangements, & Christmas tree etc.

The fragrance materials contained in the perfume composition according to the invention are compounds which are customarily used by perfumers and they were obtained from various companies such as IFF (International Flavors & Fragrances), Vigon and Givaudan.

A fragrance oil may include any one or more of extracts of natural raw materials such as essential oils, concretes, absolutes, resins, resinoids, balsams, and tinctures. Examples of natural oils used in perfume formulations are—amyris oil; angelica seed oil; angelica root oil; aniseed oil; valerian oil; basil oil; wood moss absolute; bay oil; mugwort oil; benzoin resin; bergamot oil; beeswax absolute; birch tar oil; bitter almond oil; savory oil; bucco-leaf oil; cabreuva oil; cade oil; calamus oil; camphor oil; cananga oil; cardamom oil; cascarilla oil; cassia oil; cassia absolute; castoreum absolute; cedar-leaf oil; cedarwood oil; cistus oil; citronella oil; lemon oil; copaiba balsam; copaiba balsam oil; coriander oil; costus root oil; cumin oil; cypress oil; davana oil; dill oil; dillseed oil; eau de brouts absolute; oakmoss absolute; elemi oil; tarragon oil; eucalyptus citriodora oil; eucalyptus oil; fennel oil; fir oil; galbanum oil; galbanum resin; geranium oil; grapefruit oil; guaiac wood oil; gurjun balsam; gurjun balsam oil; helichrysum absolute; helichrysum oil; ginger oil; iris root absolute; iris root oil; jasmine absolute; calamus oil; blue chamomile oil; Roman chamomile oil; carrot-seed oil; cascarilla oil; pine-needle oil; spearmint oil; caraway oil; labdanum oil; labdanum absolute; labdanum resin; lavandin absolute; lavandin oil; lavender absolute; lavender oil; lemongrass oil; lovage oil; distilled lime oil; pressed lime oil; linaloe oil: litsea cubeba oil; bay-leaf oil; mace oil; marjoram oil; mandarin oil; massoi bark oil; mimosa absolute; ambrette oil; tincture of musk; clary sage oil; myristica oil; myrrh absolute; myrrh oil; myrtle oil; clove leaf oil; clove flower oil; neroli oil; olibanum absolute; olibanum oil; opopanax oil; orange blossom absolute; orange oil; origanum oil; palmar osa oil; patchouli oil; perilla oil; Peru balsam oil; parsley leaf oil; parsley seed oil; petitgrain oil; peppermint oil; pepper oil; pimento oil; pine oil; pennyroyal oil; rose absolute; rosewood oil; rose oil; rosemary oil; Dalmatian sage oil; Spanish sage oil; sandalwood oil; celery seed oil; spike lavender oil; Japanese aniseed oil; styrax oil; tagetes oil; fir-needle oil; tea-tree oil; turpentine oil; thyme oil; Tolu balsam; Tonka absolute; tuberose absolute; vanilla extract; violet leaf absolute; verbena oil; vetiver oil; juniper oil; wine-lees oil; wormwood oil; wintergreen oil; ylang oil; hyssop oil; civet absolute; cinnamon leaf oil; cinnamon bark oil; as well as fractions thereof or constituents isolated therefrom; and combinations thereof.

Active components of many of the natural fragrances have been identified and synthesized. Derivatives of natural chemicals have been prepared and are being used as synthetic fragrances. A good example for this would be Manzanate which was used for the studies related to the release of the fragrance.

Suitable colorants include those such as dyes, pigments and lakes. Exemplary colors used in perfume formulations include: D&C Red No. 19 (e.g., CI-45170, CI-73360 or CI-45430); D&C Red No. 9 (CI 15585); D&C Red No. 21 (CI 45380); D&C Orange No. 4 (CI 15510); D&C Orange No. 5 (CI 45370); D&C Red No. 27 (CI 45410); D&C Red No. 13 (CI 15630); D&C Red No. 7 (CI 15850:1); D&C Red No. 6 (CI 15850:2); D&C Yellow No. 5 (CI 19140); D&C Red No. 36 (CI 12085); D&C Orange No. 10 (CI 45475); D&C Yellow No. 19 (CI 15985); FD&C Red #40 (CI#16035); FD&C Blue #1 (CI#42090); FD&C Yellow #5 (CI#19140); or any combinations thereof.

The compositions of the invention may optionally include “adjuvants” helping the skin and the formulations. They may include: skin penetration enhancers; emollients (e.g., isopropyl myristate, petrolatum, dimethicone oils, ester oils, mineral oils, or hydrocarbon oils); skin plumpers (e.g., palmitoyl oligopeptide), humectants (e.g., polyols, including propylene glycol, glycerin, etc.); antioxidants (e.g., BHT, ascorbic acid, sodium ascorbate, ascorbyl palmitate, beta-carotene, thiodipropionic acid, vitamin E, etc.); vitamins (e.g., tocopherol, tocopheryl acetate, etc.); alpha-hydroxy acids (e.g., glycolic acid), beta-hydroxy acids (e.g., salicylic acid); retinoids (e.g., retinoic acid, all-trans-retinoic acid, retinaldehyde, retinol, and retinyl esters such as acetates or palmitates); other anti-aging ingredients (e.g., collagen stimulators, collagenase inhibitors, elastase inhibitors); depigmenting agents (e.g., TDPA, hydroquinone, kojic acid, etc.); exfoliating agents (e.g., glycolic acid, 3,6,9-trioxaundecanedioic acid, etc.); estrogen synthetase stimulating compounds (e.g., caffeine and derivatives); compounds capable of inhibiting 5 alpha-reductase activity (e.g., linolenic acid, linoleic acid, finasteride, and mixtures thereof); and barrier function enhancing agents (e.g., ceramides, glycerides, cholesterol and its esters, alpha-hydroxy and omega-hydroxy fatty acids and esters thereof, etc.).

The compositions may also comprise a preservative or anti-microbial agent, for example, methylchloroisothiazolinone, methylisothiazolinone, methylparaben, propylparaben, phenoxyethanol, or caprylyl glycol.

There are different types of cyclodextrins available. For the purpose of this patent application, the cyclodextrin can be one or more the following —alpha cyclodextrin (alpha CD), beta cyclodextrin (beta CD), gamma cyclodextrin (gamma CD), hydroxypropyl beta cyclodextrin (HPBCD), Sulfobutyl ether beta cyclodextrin (SBEBCD), and randomly methylated beta cyclodextrin.

In some embodiments, the compositions are applied to a human integument, such as the skin of the face, hands, or body, or the hair of the scalp. It may be applied to the objects such as cloths, scarf which cover the human skin or area.

The composition can be applied as often as necessary to impart the desired fragrance intensity, cosmetic finish, color or appearance to the skin, etc. A composition according to the invention is expected to achieve extended release of fragrance oil for a long-wear period such as from about 0.1 to about 48 hours, from about 0.1 to about 24 hours or from about 0.1 to about 12 hours.

### Example 1

Table I lists ingredients of one of the perfume compositions prepared.

TABLE I
Composition of a Perfume Formulation

| Ingredient | Percent w/w |
|---|---|
| Isopropyl Myristate | 5 |
| Propylene Glycol | 15 |
| Perfumers alcohol q.s. to make | 100 |
| Jasmine India Absolute | 2.5 |
| Rose | 2.5 |

q.s. to make = quantity sufficient to make

The general process for the preparation of the perfume formulation included mixing of fragrances to a portion of Perfumers alcohol, isopropyl myristate and propylene glycol, mixing well and making up the volume with the remainder of Perfumers alcohol.

### Example II

Table II lists composition of another perfume prepared.

TABLE II
Composition of a Perfume Formulation

| Ingredient | Percent by weight |
|---|---|
| Isononyl isononanoate | 15 |
| Propylene Glycol | 15 |
| Benzyl Benzoate | 1 |
| Majantal | 0.75 |
| Rose Oxide | 1 |
| Musk | 0.5 |
| Vetiver | 0.75 |
| Bergamot | 1 |
| Frankincense | 0.5 |
| Freesia Fleuriff | 0.5 |
| Perfumers Alcohol q.s. to | 100 |

### Example 3

Table III lists the composition of a perfume, which contained a cyclodextrin.

TABLE III
Composition of a Perfume Formulation with Cyclodextrin

| Ingredient | Percent by weight |
|---|---|
| Cyclodextrin | 2 |
| Isononyl isononanoate | 12 |
| Glycerin | 16 |
| Bois De Rose | 1.6 |
| Musk fragrance oil | 2 |
| Acsantal | 1.2 |
| Fixamber | 1.2 |
| Perfumers alcohol q.s. to | 100 |

In this formulation, cyclodextrin was solid and it was very important to ensure a complete solubilization of cyclodextrin in the solvent system. The percent of cyclodextrin in this formulation can be increased suitably.

The fragrance solution in Example II was filled in three different bottles with different kinds of spray pumps. The spray pattern of each was studied by spraying the solution on a blank paper and measuring the corresponding diameter of the area. This experiment was carried out using different spraying distances. As the spraying distance increased, the diameter of the circle, encircling the spray mark on the paper, decreased. Different bottles showed different shapes of spray patterns. The amount of cyclodextrin or the moisturizer sprayed on the surface depends on the delivery volume of the particular spray pump and the size of nozzle used.

### Example 4

Table IV lists a composition of a perfume formulation, which was loaded on a powder base to prepare a scented powder delivery system.

TABLE IV
Composition of a Perfume Formulation used to make “scented”
powder

| Ingredient | Percent by weight |
|---|---|
| Cyclodextrin | 6 |
| Isopropyl Myristate | 12 |
| Propylene Glycol | 10 |
| Bois De Rose | 5 |
| Musk fragrance oil | 5 |
| Patchouli | 3 |
| Perfumers alcohol q.s. to | 100 |

A clear yellow colored solution was obtained using the composition in Table IV. The solution listed in Table IV was then loaded on a mixture of super-refined talc and calcium silicate as listed in Table V. One can use different shades of color or glitter in such formulations.

TABLE V
Composition of a Powder Perfume Delivery system

| Ingredient | Percent by weight |
|---|---|
| Formulation from Table IV | 17.4 |
| Calcium Silicate | 25.1 |
| Super refined talc | 57.5 |

Solution from Table IV was first added to calcium silicate to form a wet mass. It was mixed well to which super-refined talc was added in small portions mixing continuously. It formed a slightly wet mass, which was dried in air. It was passed through a 100 mesh sieve. After application of the scented powder on the skin, the fragrance dissipated for couple of hours. After the cessation of fragrance, few drops of water was spread over the skin. The fragrance was released again. This mimicked the sweat-activation function.

A set of experiments were designed and conducted to prove the biphasic and a long-lasting release of the model perfume from the developed composition. Manzanate, a top note, was used as a model fragrance. Many fragrances were tested to find their evaporation rates on their own in air at room temperature. Manzanate was one of the fragrances, which evaporated very fast. Manzanate, 15.7 mg was placed on a microscope slide and allowed to evaporate at room temperature. The weight of manzanate was determined over time. The rate of evaporation was calculated to be 1.14 mg/min. It was considered as a top note. Another noteworthy top note observed was eucalyptus oil with the evaporation rate of 1.01 mg/min. Ethyl isovalerate showed fastest evaporation with a rate of 3.12 mg/min. Some of base notes showed evaporation rates as low as 0.01 mg/min.

In most of the studies on perfumes, gas chromatography was used as the analytical technique to determine the concentrations of fragrances. In this experiment, high performance liquid chromatography (HPLC) was used to determine the concentration of manzanate in the perfume formulation. Phenomenex Luna 5 μm Phenyl-Hexyl 150×4.60 mm column was used for this assay. Mobile Phase A contained 0.1% trifluoroacetic acid in water and Mobile Phase B contained methanol. The wavelength of detection used was 210 nm. One of the criteria for the selection of the model drug was its absorptivity so that it can analyzed by HPLC.

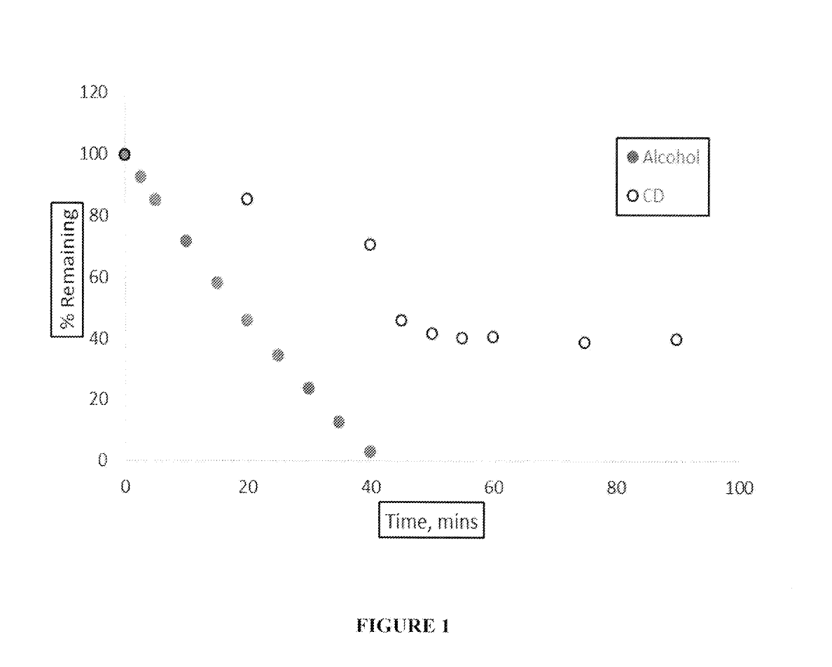

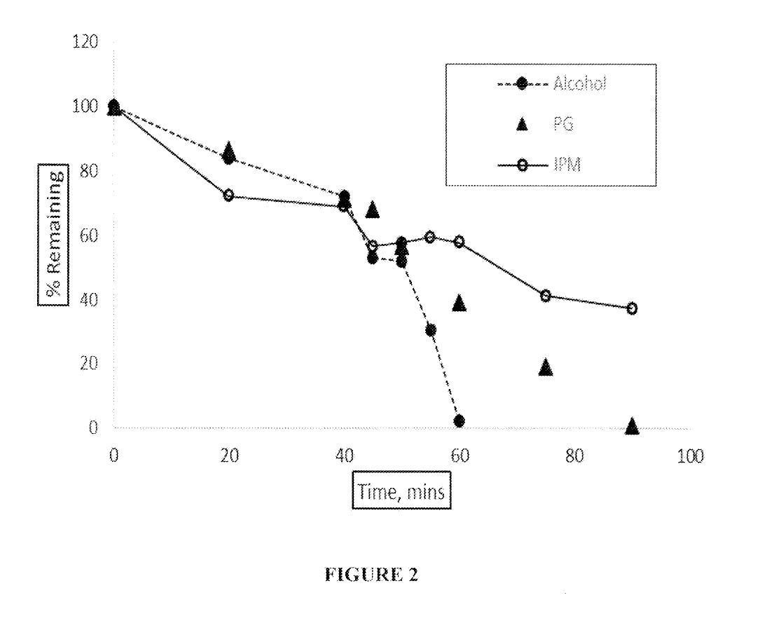

Various formulations of manzanate were prepared in perfumer's alcohol along with other excipients. The perfume formulations, 100 μL, were placed in a set of 10 mL beakers and the perfume was allowed to evaporate at room temperature. At various predetermined times, a diluent was added to respective beakers to dissolve manzanate, which had not evaporated from the formulations. The concentrations of manzanate were determined by HPLC. The percent manzanate remaining values were determined and plotted against time. In another experiment, Perfumers alcohol 100 μL without manzanate or other excipients was allowed to evaporate and the weight of alcohol remaining was calculated by loss of weight (FIG. 1). It provided the time to evaporate alcohol from the formulations, which was about 42 minutes. In the presence of an excipient, two distinct sections in the “% manzanate remaining values” were observed—A and B. Section A ended at ˜42 minutes when all the alcohol evaporated. Different formulations of manzanate were prepared as listed in Table VI. Percent manzanate remaining values over time were determined by the HPLC analysis (FIGS. 1 and 2). Slope values of lines in sections A and B were determined and have been listed in Table VI.

TABLE VI
Composition of Manzanate and the Slope Values of Sections A and B.

| | Slope | |
|---|---|---|
| Composition | for Section A | Slope for Section B |
| Manzanate in Perfumer's alcohol | 0.692 | 3.247 |
| Manzanate in Perfumer's alcohol + | 0.711 | 1.387 |
| Propylene glycol | | |
| Manzanate in Perfumer's alcohol + | 0.701 | 0.674 |
| Isononyl Isononanoate | | |
| Manzanate in Perfumer's alcohol + | 0.838 | 0.618 |
| Isopropyl Myristate | | |
| Manzanate in Perfumer's alcohol + | 0.884 | 0.0407 |
| Cyclodextrin | | |

It is clear that the slope values were similar for Section A in all the formulations. Thus, a portion (30 to 40%) of manzanate evaporated along with alcohol (FIGS. 1 and 2). Once all the alcohol was evaporated at about 42 minutes, the rate of evaporation of manzanate was governed by the remaining excipients (FIG. 2). In the absence of excipients, the rate of evaporation of manzanate was governed by the intrinsic property of manzanate. This is true for all the fragrances which can be used in the perfume formulations. The slope was highest in the absence of any excipient showing fast evaporation of manzanate. Addition of propylene glycol decreased the rate of evaporation by nearly half. IPM and Isononyl isononanoate decreased the evaporation further to one fifth of the initial rate. The data was clear as depicted in FIG. 2. Manzanate being a top note has an evaporate rate very fast. Most of the base notes tested showed evaporation rates of 0.05 mg/min compared to the rate of 1.12 mg/min for manzanate. One can argue that manzanate was changed from top note to middle note by the addition of IPM and Isononyl isononanoate. Thus, manzanate or other top notes will be retained on the surface over several hours. In the presence of IPM, 40% of manzanate remained in the beaker even after 1.5 hours. The slope value of 0.0407 was observed for section B when CD was added to the carrier alcohol (FIG. 1). It suggested that manzanate was retained in CD and evaporated extremely slowly.

FIGS. 1 and 2 demonstrate the biphasic release of the model compound. For the purpose of this patent application, a “biphasic release” is defined as having two distinct regions in the release profile of the perfume having significantly different release or evaporation rates. When one has a mixture of fragrances, there will be separate biphasic release profiles for each fragrances. The release rates will depend on the hydrophobicity of the fragrance, their evaporation rate as is, the kind of emollients or moisturizing agents added etc. If the two fragrances interact with each other, that will also alter the evaporation rates of the individual fragrances.

In another set of experiments, 50, 100, 150, 200 and 250 μL of manzanate solution in alcohol and CD were placed in 10-mL beakers. The beakers were placed in a 25° C. stability chamber with forced air circulation. The manzanate formulation evaporated completely in 3 hours. The amount of manzanate remaining in each beaker was determined. A linear correlation was observed between the amounts of manzanate remaining versus the volume of solution added. It means, manzanate which was not embedded in the cyclodextrin evaporated from all the samples. Knowing the molecular weights of manzanate and cyclodextrin, it was observed that one mole of manzanate complexes with one mole of cyclodextrin. This complexation constant (number of moles per mole of cyclodextrin) will be different for each fragrance. Also, the binding constant will be different for each fragrance—stronger the binding, longer the perfume stay in the cyclodextrin bucket. In the mixture of fragrances, there will be competition between the fragrances to occupy the cavity in the cyclodextrin. It may be possible that fragrance A may occupy the CD cavity at time zero, but will be replaced by fragrance B when an equilibrium is attained.

Perfume formulation such as in the Example 3 can be tested for the sweat-activation. A typical test involves the following steps. During usage, one would spray perfume on the body. Cyclodextrin forms a thin film. The free perfume will evaporate over time, leaving behind the film of cyclodextrin with perfume locked in it. When the user sweats, being water-soluble, cyclodextrin dissolves. Once in a soluble form, the molecules of cyclodextrin molecules become mobile and flexible. Because there is higher concentration of perfume in the cyclodextrin bucket and less in the sweat, a chemical gradient is established. It helps to draw out or diffuse the perfumes out of cyclodextrin cavity to the sweat. Once in the aqueous sweat, the perfume is released in air and more fragrance molecules diffuse out of cyclodextrin cavity. This is the mechanism of sweat activation. In places where one does not sweat, the effect can be tested by applying a small amount of water on the skin where the sweat-activated perfume is applied.

Perfume composition from Example III was sprayed, allowed to evaporate and then small amount of water was applied to mimic sweating. The fragrance smell could be detected very easily. This proved the sweat-activation of the system.

The invention described and claimed herein is not to be limited to examples disclosed. Any equivalent embodiments are intended to be within the scope of this invention and can be understood easily to those skilled in the art.

For example, another variation of the perfume composition can be a perfume delivery system with a double-action. The two actions could be moisturizing and long-lasting and it would be devoid of sweat-activation. One may add either a moisturizing agent or an emollient in the perfume along with cyclodextrin creating another variation.

In conclusion, a novel type of perfume delivery system has been invented, which has unique and useful properties. The system is moisturizing, emollient, long-lasting and sweat-activated. By selection of appropriate excipients, one can select one or more properties in a perfume formulation. For example, one can make a perfume, which is only sweat-activated or a system, which is emollient and sweat-activated. This perfume delivery system produces biphasic release profiles for each of the fragrances added to the formulation. This is a perfume delivery system and not restricted to particular a mixture of fragrances. The system can be used to deliver any combination of fragrances. The liquid double-action or triple-action perfume delivery system can be loaded on a suitable powder base to produce a scented powder. This scented powder formulation also showed sweat-activation property. One can incorporate various colors and glitters, which are matching with the fragrances included in the formulation.

What is claimed is:

1. A triple-action perfume delivery system with a bimodal release profile including a composition comprising: (a) from about 0.1% to 30% of moisturizing agent; (b) from about 0.1% to 30% of an emollient; (c) from about 0.1% to 15% of a cyclodextrin; (d) from about 10% to 90% of a solvent; (e) from about 0.1% to 20% of adjuvants; and (f) from about 0.1% to 25% of a plurality of fragrances.

2. A system in claim 1 wherein the triple-action comprises of moisturing/emollient, long-lasting and sweat activated effects.

3. A system in claim 1 which is applied to human inegument and non-living surfaces.

4. A system in claim 1 which is loaded on a powder base and resultant composition is applied to human inegument and non-living surfaces.

5. A system in claim 1 wherein the said moisturizing agent is at least one member selected from the group consisting of, but not limited to, propylene glycol, polyethylene glycol 400, butylene glycol, glyceryl triacetate, sorbitol, glycerin and mixtures thereof.

6. A system in claim 1 wherein the said emollient is at least one member selected from the group consisting of, but not limited to, isononyl isononanoate, isopropyl myristate, cyclomethicone and mixtures thereof.

7. A system in claim 1 wherein the said cyclodextrin is at least one member selected from the group consisting of, but not limited to, alpha cyclodextrin, beta cyclodextrin, gamma cyclodextrin, hydroxypropyl beta cyclodextrin, sulfobutyl ether beta cyclodextrin, and randomly methylated beta cyclodextrin and mixtures thereof.

8. A system in claim 1 wherein the said solvent is at least one member selected from the group consisting of, but not limited to, perfumers alcohol, 1,2-hexanediol, 1,2-heptanediol, volatile silicones, water and mixtures thereof.

9. A system in claim 1 in which the adjuvants are selected from the group consisting of, but not limited to, fixatives, preservatives, antioxidants, colors, glitter, and mixtures thereof.

10. A double-action perfume delivery system with a bimodal release profile including a composition comprising: (a) from about 0.1% to 30% of moisturizing agent; (b) from about 0.1% to 30% of an emollient; (c) from about 10% to 90% of a solvent; (d) from about 0.1% to 20% of adjuvants; and (e) from about 0.1% to 25% of a plurality of fragrances.

11. A system in claim 10 wherein the double-action comprises of moisturing/emollient, and long-lasting effects.
